(19) United States
(12) Patent Application Publication
(10) Pub. No.: US 2021/0278404 A1
(43) Pub. Date: Sep. 9, 2021

(54) COMPETITIVE LATERAL FLOW ASSAY

(71) Applicant: CORNELL UNIVERSITY, Ithaca, NY (US)

(72) Inventors: David ERICKSON, Ithaca, NY (US); Seoho LEE, Ithaca, NY (US); Saurabh MEHTA, Ithaca, NY (US); Sasank Sai VEMULAPATI, Ithaca, NY (US); Elizabeth Grace REY, Ithaca, NY (US); Dakota O'DELL, Ithaca, NY (US)

(21) Appl. No.: 17/326,472

(22) Filed: May 21, 2021

Related U.S. Application Data
(63) Continuation of application No. 15/776,895, filed on May 17, 2018.
(60) Provisional application No. 62/256,946, filed on Nov. 18, 2015.

(51) Int. Cl. G01N 33/558; G01N 33/82

(52) U.S. Cl. CPC G01N 33/558; G01N 33/82

(57) ABSTRACT

A competitive diagnostic assay strip includes a first layer on a substrate for receiving, absorbing, and filtering a liquid sample. A second layer is on the substrate downstream of the first layer and includes a specific binding partner of a target molecule that will bind to the target molecule and produce a complex. A third layer is on the substrate downstream of the second layer and includes test and control regions. The test region has target molecules which will bind to and immobilize the complex in the test region. The control region has a moiety which will non-specifically bind to and immobilize the binding partner in the control region. In one embodiment, a spacer layer is on the substrate downstream of the second layer and designed to substantially stop flow of material from the second layer within the spacer layer to provide added mixing and incubation time for formation of the complex. In another embodiment, an extraction layer is on the substrate downstream of the first layer and upstream of the second layer. Methods of use are also disclosed.

This application is a continuation application of U.S. patent application Ser. No. 15/776,895 filed May 17, 2018, which is a national stage application under 35 U.S.C. § 371 of PCT Application No. PCT/US2016/062828, filed Nov. 18, 2016, which claims the priority benefit of U.S. Provisional Patent Application Ser. No. 62/256,946, filed Nov. 18, 2015, which is hereby incorporated by reference in its entirety.

### GOVERNMENT FUNDING

This invention was made with government support under 1430092 awarded by National Science Foundation. The government has certain rights in the invention.

### FIELD OF THE INVENTION

The present invention relates to a competitive lateral flow assay and methods of use thereof.

### BACKGROUND OF THE INVENTION

Vitamin B₁₂ deficiency is the leading cause of cognitive decline in the elderly and is associated with increased risks of several acute and chronic conditions including anemia. The primary source of vitamin B₁₂ is diary food intake and as such, the deficiency has been reported to be the highest among the vegetarian and vegan populations. Although deficient levels of vitamin B₁₂ can usually be improved by consuming supplements and/or increasing diary food intake at an earlier stage, most are unaware of their condition due to the lack of a simple diagnostics system for vitamin B₁₂.

Similarly, over 50% of U.S. adults are thought to have insufficient levels of vitamin D. Vitamin D deficiency is strongly correlated to a number of serious pathologies, including osteoporosis, rickets, and multiple sclerosis. Although deficiencies are reversible through changes in diet, they are often asymptomatic early in their course, meaning that many affected individuals do not know their status without diagnostic testing.

The development of a lateral flow B₁₂ test has been a challenge due to the extremely low detection range that is required for B₁₂ deficiency tests. The recommended cutoffs for vitamin B₁₂ deficiency are defined in humans by blood vitamin B₁₂ below 500 pg/ml. However, commercial lateral flow assays have only been demonstrated for detection of targets in the sub-μg/ml to μg/ml range. Vitamin D is similarly difficult to measure due to a low deficiency cut-off of 20 ng/ml and the presence of the vitamin D binding protein that must be removed before an assay. The binding protein removal is especially critical, as this separation step often requires organic solvents and significant sample processing. Due to these restrictions, there is currently no low-cost, quantitative test for point-of-need vitamin B₁₂ or vitamin D detection.

Over the past decades, lateral flow immunochromatography assays have been widely adopted for diagnosing various diseases and medical conditions in point-of-care settings. These assays are rapid, simple, and produce colorimetric signals that can be interpreted by untrained personnel. The development of lateral flow type assays for vitamin B₁₂ and D could greatly facilitate the deployment of these tests by making them more accessible.

The present invention is directed to overcoming these and other deficiencies in the art.

### SUMMARY OF THE INVENTION

One aspect of the present invention relates to a competitive diagnostic assay strip for detection of a target molecule in a sample. The strip includes an elongate substrate extending between a first end at which the sample is applied to the strip and a second end at which results of the assay can be assessed. A first layer is supported on the elongate substrate proximate to the first end of the strip for receiving, absorbing, and filtering a liquid sample. A second layer is supported on the elongate substrate, distal from the first and the second ends of the strip, and downstream of the first layer. The second layer includes a mobile labeled specific binding partner of the target molecule, where the labeled specific binding partner will bind specifically to the target molecule passing from the first layer through the second layer toward the second end of the strip and produce a complex of the target molecule bound to the labeled specific binding partner. A third layer is supported on the elongate substrate proximate to the second end of the strip and downstream of the second layer. The third layer includes a test region and a control region separated from the test region. The test region has immobilized target molecules which will specifically bind to the complex and immobilize it in the test region and the control region has an immobilized moiety which will non-specifically bind to the labeled specific binding partner of the target molecule and immobilize it in the control region. A first spacer layer is supported on the elongate substrate downstream of the second layer. The first spacer layer is designed to substantially stop flow of material from the second layer within the first spacer layer until a further fluid flow is provided to achieve added mixing and incubation time for formation of the complex of the target molecule bound to the labeled specific binding partner.

A further aspect of the present invention relates to a competitive diagnostic assay strip for detection of a target molecule in a sample. The strip includes an elongate substrate extending between a first end at which the sample is applied to the strip and a second end at which results of the assay can be assessed. A first layer is supported on the elongate substrate proximate to the first end of the strip for receiving, absorbing, and filtering a liquid sample. A second layer is supported on the elongate substrate, distal from the first and the second ends of the strip, and downstream of the first layer. The second layer includes a mobile labeled specific binding partner of the target molecule, where the labeled specific binding partner will bind specifically to the target molecule passing from the first layer through the second layer toward the second end of the strip and produce a complex of the target molecule bound to the labeled specific binding partner. A third layer is supported on the elongate substrate proximate to the second end of the strip and downstream of the second layer. The third layer includes a test region and a control region separated from the test region, where the test region has immobilized target molecules which will specifically bind to the complex and immobilize it in the test region and the control region has an immobilized moiety which will non-specifically bind to the labeled specific binding partner of the target molecule and immobilize it in the control region. An extraction layer is supported on said elongate substrate downstream of the first layer and upstream of the second layer, the extraction layer being designed to permit recovery of the target molecule with an extracting agent.

Another aspect of the present invention relates to a diagnostic assay cartridge including the competitive diagnostic assay strip according to the present invention. The diagnostic assay cartridge includes an elongate housing having walls defining a chamber in which the competitive diagnostic assay strip is positioned. The cartridge extends between a first end proximate to the first end of the elongate substrate, where the sample is inserted through an inlet passage in a wall of the housing and into the chamber, and a second end proximate to the second end of the elongate substrate at which results of the assay can be assessed.

Yet another aspect of the present invention relates to a method of conducting a diagnostic assay. The method includes providing a competitive diagnostic assay strip in accordance with the present invention. A sample is applied to the first layer. A buffer is applied to the first layer after applying the sample to the first layer, whereby the buffer causes flow of material from the first end of the elongate substrate to the second end of the elongate substrate. The test and control regions in the third layer are analyzed to determine whether the target material is present in the sample and/or what quantity of the target material is present.

The present invention provides completive diagnostic assay strips and methods of conducting a diagnostic assay that allow for blood sample processing and key reagent storage on-chip. This provides a sample-in-answer-out diagnostic format that is suitable for point-of-care diagnostic applications. This may be utilized for target materials previously incapable of being measured using such techniques, such as vitamins B₁₂ and D. The present invention advantageously provides: (1) a spacer pad that provides increased incubation time; (2) amplification of the obtained signal to decrease overall assay time; (3) an extraction buffer for separating binding proteins from a target molecule that is compatible with a lateral flow format; and (4) an improved blood filtration design to increase plasma yield from finger stick blood. The present invention provides for low-cost, quantitative tests for point-of-need detection of difficult to detect target materials, such as vitamins B₁₂ and D, directly from finger prick blood.

### BRIEF DESCRIPTION OF THE DRAWINGS

FIGS. 1A-1C are perspective views of an embodiment of a competitive diagnostic assay strip of the present invention, where vitamin B₁₂ is detected. FIG. 1A further illustrates a cartridge that may house the competitive diagnostic assay strip.

FIG. 2 is a perspective view of the competitive diagnostic assay strip of the present invention including an extraction layer.

FIG. 3 is a perspective view of the competitive diagnostic assay strip illustrated in FIG. 2 including an additional spacer layer.

FIG. 4 is a perspective view of another embodiment of a competitive diagnostic assay strip of the present invention.

FIG. 5 is a flow chart of a method of conducting a diagnostic assay in accordance with the present invention, where vitamin B₁₂ is detected.

FIG. 6 is a flow chart of another method of conducting a diagnostic assay in accordance with the present invention, where vitamin D is detected.

FIGS. 7A-7D illustrate test results for a silver-enhanced B₁₂ diagnostic assay with an optimized condition of 0.35 mg/ml vitamin B₁₂-BSA-0.06OD gold anti-vitamin B₁₂. FIG. 7A provides captured images of the test and control line regions on the test strip. FIGS. 7B-7C illustrate test and control line signals detected for 0 and 750 pg/ml of vitamin B₁₂ standard samples. FIG. 7D is calibration curve of T/C ratio vs. vitamin B₁₂ concentration from the captured images.

FIGS. 8A and 8B illustrate 0 vs 1 ng/ml vitamin B₁₂ differentiation with another optimized condition of 0.325 mg/ml vitamin B₁₂-BSA-0.053 OD gold anti-vitamin B₁₂. FIG. 8A illustrates the captured image of the test and control lines and detected signals for a 0 ng/ml standard vitamin B₁₂ sample, and FIG. 8B illustrates the captured image of the test and control lines and detected signals for a 1 ng/ml standard vitamin B₁₂ sample.

FIGS. 9A-9C illustrate human trial results including the captured image of the test and control lines and detected signals for a participant with vitamin B₁₂ levels of 167 pg/ml (FIG. 9A), a participant with vitamin B₁₂ levels of 175 pg/ml (FIG. 9B), and a participant with vitamin B₁₂ levels of >1000 pg/ml (FIG. 9C)

FIGS. 10A and 10B illustrate the effect of the extraction buffer for a vitamin D assay including the raw images and processed signals for a serum sample without extraction buffer (FIG. 10A) and with the extraction buffer (FIG. 10B)

FIGS. 11A-11C illustrate a quantification of results from serum human trials for a vitamin D assay including captured images and processed signals for three concentrations of 25(OH)D for 15 ng/ml (FIG. 11A), 24 ng/ml (FIG. 11B), and 42 ng/ml (FIG. 11C).

FIG. 12 illustrates a full calibration curve for the serum human trials for a vitamin D analysis using a competitive diagnostic assay strip of the present invention.

### DETAILED DESCRIPTION

The present invention relates to competitive lateral assays. More specifically, the present invention relates to a competitive diagnostic assay strip, a diagnostic assay cartridge including the competitive diagnostic assay strip, and a method of conducting a diagnostic assay.

One aspect of the present invention relates to a competitive diagnostic assay strip for detection of a target molecule in a sample. The strip includes an elongate substrate extending between a first end at which the sample is applied to the strip and a second end at which results of the assay can be assessed. A first layer is supported on the elongate substrate proximate to the first end of the strip for receiving, absorbing, and filtering a liquid sample. A second layer is supported on the elongate substrate, distal from the first and the second ends of the strip, and downstream of the first layer. The second layer includes a mobile labeled specific binding partner of the target molecule, where the labeled specific binding partner will bind specifically to the target molecule passing from the first layer through the second layer toward the second end of the strip and produce a complex of the target molecule bound to the labeled specific binding partner. A third layer is supported on the elongate substrate proximate to the second end of the strip and downstream of the second layer. The third layer includes a test region and a control region separated from the test region. The test region has immobilized target molecules which will specifically bind to the complex and immobilize it in the test region and the control region has an immobilized moiety which will non-specifically bind to the labeled specific binding partner of the target molecule and immobilize it in the control region. A first spacer layer is supported on the elongate substrate downstream of the second layer. The first spacer layer is designed to substantially stop flow of material from the second layer within the first spacer layer until a further fluid flow is provided to achieve added mixing and incubation time for formation of the complex of the target molecule bound to the labeled specific binding partner.

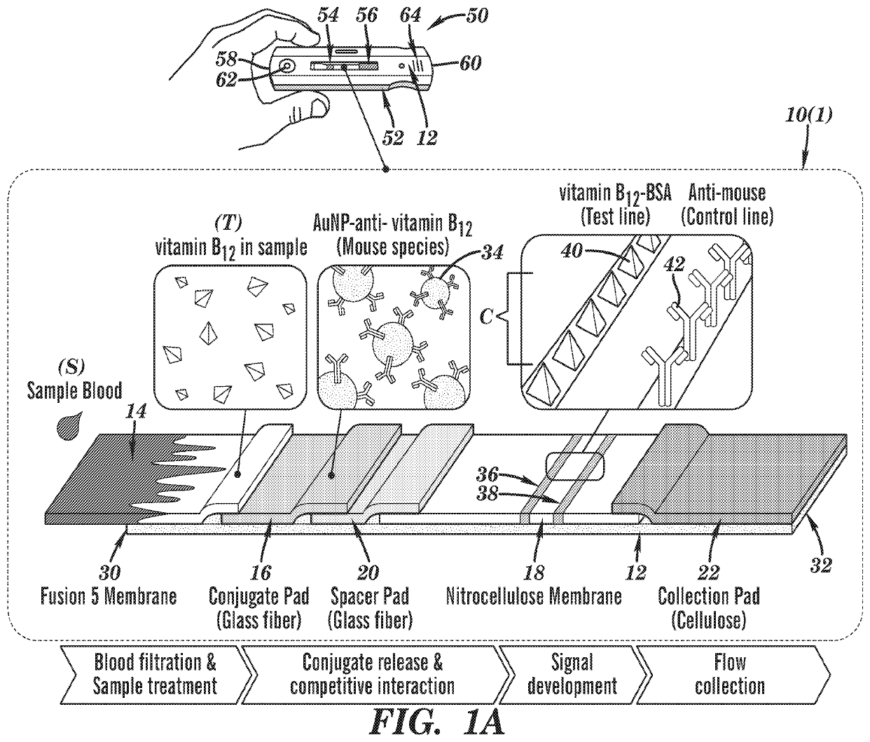

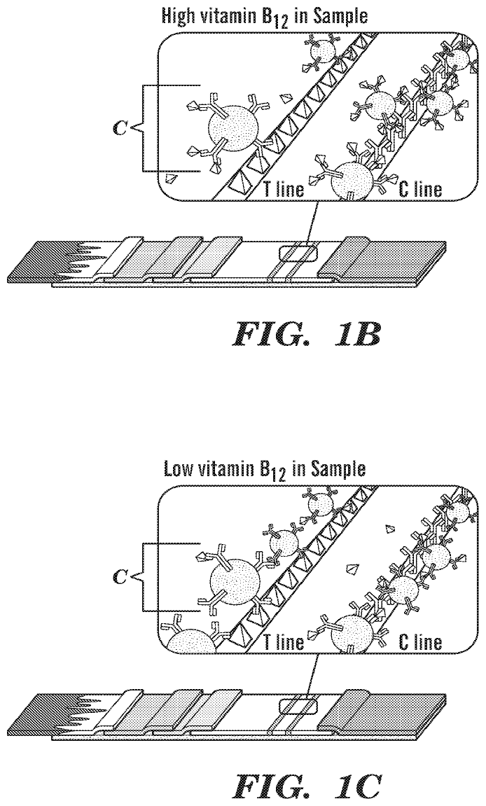

FIGS. 1A-1C are perspective views of a first embodiment of competitive diagnostic assay strip 10(1) of the present invention. Competitive diagnostic assay strip 10(1) may be utilized for detection of a target molecule (T) in a sample (S), such as a blood sample, a plasma sample, a serum sample, a urine sample, a saliva sample, a sweat sample, cerebral spinal fluid, or tears.

The target molecule (T) may be selected from the group consisting of vitamins, such as vitamin B₁₂ or vitamin D, micronutrients, genetic biomarkers, such as DNA or RNA, carbohydrates, and proteins.

Exemplary vitamin target molecules, include, but are not limited to, vitamin A, vitamin D, vitamin E, vitamin B₁ (thiamine), vitamin B₂ (riboflavin), vitamin B₃ (niacin), vitamin B₆, vitamin B₇ (biotin), folate (folic acid and vitamin B₉), vitamin B₁₂ (cyanocobalamin), vitamin C, and pantothenic acid.

Suitable micronutrients include, but are not limited to, iron, cobalt, zinc, manganese, copper, iodine, selenium, molybdenum, and chromium.

Exemplary target nucleic acid molecules include, but are not limited to, viral, bacterial, and tumor-associated nucleic acid molecules.

Carbohydrates may be selected from the group consisting of monosaccharides (such as glucose, galactose, fructose, xylose), disaccharides (such as sucrose, lactose, maltose, trehalose), oligosaccharides (such as malto-dextrin, raffinose, and stachyose), and polysaccharides (such as amylose, amylopectin, cellulose, hemicellulose, pectins, and hydrocolloids).

Suitable target protein molecules include, but are not limited to, antibodies, antibody fragments, epitopes, hormones, neurotransmitters, cytokines, growth factors, cell recognition molecules, cell receptors, bacterial proteins, viral proteins, toxins, prions, disease-associated proteins, and fragments thereof

As shown in FIG. 1A, competitive diagnostic assay strip 10(1) may be housed in diagnostic assay cartridge 50 as described in further detail below.

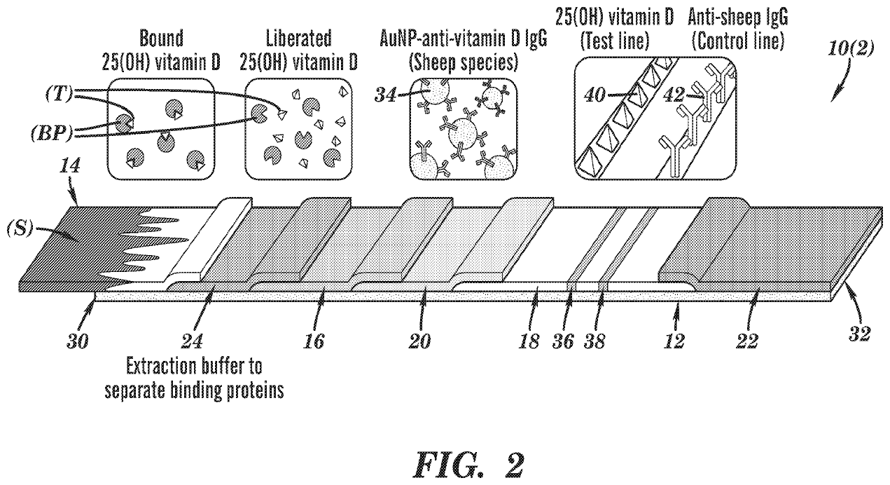

Referring again to FIGS. 1A-1C, competitive diagnostic assay strip 10(1) includes substrate 12, first layer 14, second layer 16, third layer 18, first spacer layer 20, and collection layer 22. Competitive diagnostic assay strip 10(1) may also include other types or numbers of layers. Non-limiting examples of additional elements and configurations include those described below with respect to FIGS. 2-4 discussed infra.

In one example, competitive diagnostic assay strip 10(1) is configured to be utilized in conjunction with a smartphone as described in U.S. Patent Application Publication No. 2016/0080548 and PCT Patent Application PCT/US14/12263, the disclosures of which are hereby incorporated by reference in their entirety herein. Specifically, competitive diagnostic assay strip 10(1) may be inserted into a smartphone accessory that provides for analysis of competitive diagnostic assay strip 10(1). The smartphone receives image data from the smartphone accessory to provide a quantification of the results of the diagnostic assay.

Substrate 12 has an elongate form extending between first end 30 at which the sample (S) is applied to competitive diagnostic assay strip 10 and second end 32 at which results of the assay can be assessed. Substrate 12 is configured to support the various layers of competitive diagnostic assay strip 10(1) as described below. In one example, substrate 12 is a Flow Plus 180 Membrane Card (EMD Millipore, Billerica, Mass.) with a 2 mm clear polyester film backing to which the layers described below may be adhesively attached, by way of example, although other suitable substrates may be utilized.

First layer 14 is supported on substrate 12 proximate to first end 30 of competitive diagnostic assay strip 10(1). First layer 14 provides a membrane for receiving, absorbing, and filtering a liquid sample (S), such as capillary blood from a finger stick. First layer 14 is made of a material selected from the group consisting of cellulose membranes, polyester matrix, glass fiber, and polysulfone membranes. Examples of suitable materials for first layer 14 include a Fusion 5 membrane produced by GE Whatman, St. Louis, Mo., or an FR-1 membrane produced by MDI

Membrane Technologies, India. In one example, first layer 14 is vertically stacked relative to substrate 12 to enhance receiving, absorbing, and filtering the liquid sample (S) as described in U.S. Patent Application Publication No. 2016/0080548 and PCT Patent Application PCT/US14/12263, the disclosures of which are hereby incorporated by reference in their entirety herein.

Second layer 16 provides a conjugate pad for storing antibody conjugates. Second layer 16 is supported on substrate 12, distal from both first end 30 and second end 32 of substrate 12 and downstream of first layer 14 such that sample (S) may flow from first layer 14 to second layer 16. A portion of second layer 16 is overlapped by first layer 14 to enhance fluid flow between first layer 14 and second layer 16. In one example, second layer 16 is made of glass fibers, although other suitable materials may be utilized. Second layer 16 includes mobile labeled specific binding partner 34 located therein. Mobile labeled specific binding partner 34 is selected to be a binding partner of the target molecule (T), such that mobile labeled specific binding partner 34 will bind specifically to the target molecule (T) when the target molecule (T) passes from first layer 14 through second layer 16 toward second end 32 of competitive diagnostic assay strip 10(1) and produce a complex (C) of the target molecule (T) bound to mobile labeled specific binding partner 34, as shown in FIGS. 1B and 1C. As shown in FIGS. 1A-1C, in one example, mobile labeled specific binding partner 34 is an AuNP-anti-vitamin B₁₂ conjugate that will specifically bind to vitamin B₁₂ target molecules, although other binding partners may be utilized for other target molecules. Exemplary specific binding partners include, but are not limited to, nucleic acids such as DNA or RNA aptamers, DNA or RNA ligands, and proteins such as antibodies, antibody fragments, peptide aptamers, and ligands. The label for mobile labeled specific binding partner 34 is selected from the group consisting of carbon nano-particles, metallic nano-particles, magnetic nano-particles, fluorophores, quantum dots, and chemiluminescent particles. In this example, the label for mobile labeled specific binding partner 34 are gold-nanoparticles (AuNPs).

Third layer 18 is supported on substrate 12 proximate to second end 32 of competitive diagnostic assay strip 10 and downstream of second layer 16. Third layer 18 is made of a material selected from the group consisting of cellulose and nitrocellulose, although other suitable materials may be utilized. Third layer 18 includes test region 36 and control region 38 separated from test region 36.

Test region 36 has immobilized target molecules 40 located therein which will specifically bind to the complex (C) and immobilize the complex (C) in test region 36 as shown in FIGS. 1B and 1C. The immobilized target molecules may include vitamins, micronutrients, nucleic acids, carbohydrates, proteins, and peptides, as described above. In this example, the immobilized target molecules are vitamin B₁₂ molecules. In one example, test region 36 includes a plurality of different immobilized target molecules 40. Control region 38 has an immobilized moiety 42 located therein which will non-specifically bind to mobile labeled specific binding partner 34 of the target molecule and immobilize it in control region 38. Immobilized moiety 42 may include a species specific anti-immunoglobulin reagent such as an anti-mouse, anti-horse, anti-bovine, anti-rat, anti-sheep, anti-goat, and anti-chicken antibody or various aptamers including, but not limited to, non-specific protein and nucleic acid aptamers. Although test region 36 and control region 38 are described, a plurality of test regions and control regions including different immobilized target molecules and immobilized moieties thereon can be included on third layer 18 to provide a multiplexed assay for different target molecules. In one example, test region 36 and control region 38 include a signal enhancement solution, such as a silver enhancement solution to allow for better imaging of test region 36 and control region 38 and to provide for lower limits of detection, although the signal enhancement solution may alternatively be added to test region 36 and control region 38 by a user.

First spacer layer 20 is supported on substrate 12 downstream of second layer 16. In this example, first spacer layer 20 is disposed between second layer 16 and third layer 18 for receiving the sample (S) along with complex (C) formed in second layer 16, although first spacer layer 20 may be supported in other locations along substrate 12. A portion of first spacer layer 20 overlaps third layer 18 to provide fluid flow from first spacer layer 20 to third layer 18. In one example, first spacer layer is made of high-capacity glass fibers, although other suitable materials may be utilized. In one example, first spacer layer 20 is formed from product number GFDX 103000 produced by EMD Millipore, Billerica, Mass.

First spacer layer 20 is designed to substantially stop flow of the material received from second layer 16 within first spacer layer 20 until a further fluid flow is received. By way of example, first spacer layer 20 has a high thickness, high material weight, and a large surface area to maximize the volume capacity of first spacer layer 20. The volume capacity is configured such that the input volume of the sample (S) will be insufficient to overflow first spacer layer 20 in order to reach third layer 18 without further user interaction. In one example, first spacer layer 20 has a thickness of about 0.43 mm and a weight of 75 g/m². First spacer layer 20 may be cut to dimensions of about 10 mm×4 mm, although the dimensions of first spacer layer 20 may be designed depending on the intended input volume of the sample (S), e.g., a length of first spacer layer 20 may be increased to accommodate a higher input volume, or decreased for a lower input volume. In order to restart the fluid flow between first spacer layer 20 and third layer 28, the user must introduce a volume of running buffer that fills the remaining volume of first spacer layer 20 and then drives the sample (S) to third layer 18. The dimensions of first spacer layer 20, and in particular the volume capacity, determine the amount of additional fluid, such as a running buffer, that must be added to overflow first spacer layer 20 to deliver the sample (S) to third layer 18 for testing. The ability to substantially stop the sample (S) in first spacer layer 20 allows for arbitrary incubation periods that may be determined by the user as described in further detail below. The delay provides added mixing and incubation time for formation of the complex (C) of the target molecule (T) bound to mobile labeled specific binding partner 34, and allows the user to actively control the incubation period.

Collection layer 22 is supported on substrate 12 downstream of third layer 18 and proximate to second end 32 of competitive diagnostic assay strip 10(1). Collection layer 22 provides an absorbent pad that is designed to receive materials passing through competitive diagnostic assay strip 10(1) to collect the sample (S) for test completion. Collection layer 22 is made of a material selected from the group consisting of cellulose membranes, polyester matrix, glass fiber, and polysulfone membranes.

Another aspect of the present invention relates to a competitive diagnostic assay strip for detection of a target molecule in a sample. The strip includes an elongate substrate extending between a first end at which the sample is applied to the strip and a second end at which results of the assay can be assessed. A first layer is supported on the elongate substrate proximate to the first end of the strip for receiving, absorbing, and filtering a liquid sample. A second layer is supported on the elongate substrate, distal from the first and the second ends of the strip, and downstream of the first layer. The second layer includes a mobile labeled specific binding partner of the target molecule, where the labeled specific binding partner will bind specifically to the target molecule passing from the first layer through the second layer toward the second end of the strip and produce a complex of the target molecule bound to the labeled specific binding partner. A third layer is supported on the elongate substrate proximate to the second end of the strip and downstream of the second layer. The third layer includes a test region and a control region separated from the test region, where the test region has immobilized target molecules which will specifically bind to the complex and immobilize it in the test region and the control region has an immobilized moiety which will non-specifically bind to the labeled specific binding partner of the target molecule and immobilize it in the control region. An extraction layer is supported on said elongate substrate downstream of the first layer and upstream of the second layer, the extraction layer being designed to permit recovery of the target molecule with an extracting agent.

FIG. 2 illustrates an alternative embodiment of a competitive diagnostic assay strip of the present invention. Competitive diagnostic assay strip 10(2) is the same as competitive diagnostic assay strip 10(1) except as described below. Specifically, competitive diagnostic assay strip 10(2) includes extraction layer 24 and optional first spacer layer 20 and optional second spacer layer 26. As illustrated in FIG. 2, competitive diagnostic assay strip 10(2) may be utilized to perform an assay to measure the amount of 25(OH)D (vitamin D) in a blood sample, although competitive diagnostic assay 10(2) may be utilized to measure other target molecules (T). One of the main challenges in translating vitamin D diagnostics to a lateral flow is the presence of the binding protein (BP). Vitamin D, being fat-soluble and hydrophobic does not travel freely through the blood stream, but is instead predominantly transported by the vitamin D binding protein (BP). In order to quantify vitamin D concentrations, it is necessary to first release the vitamin D from the binding protein (BP). Although vitamin D is described, competitive diagnostic assay 10(2) could be utilized with other target molecules (T) that benefit from an extraction step. In this example, second layer 16 includes AuNP-anti-vitamin D IgG as mobile labeled specific binding partner 34.

Extraction layer 24 is supported on substrate 12 downstream of first layer 14 and upstream of second layer 16. Extraction layer 24 is made of a material selected from the group consisting of glass fibers and cellulose, although other suitable materials may be utilized. Extraction layer 24 is designed to permit recovery of the target molecule (T) with extracting agent 46, such as an extracting buffer. In one example, extracting agent 46 comprises dimethyl sulfoxide and an acetate buffer for separating an analyte, such as vitamin D, from a binding protein. The extraction layer 24 holds extraction agent 46 so that sample (S) passed through first layer 14 can interact with extracting agent 46, although an extracting buffer may be separately applied to extraction layer 24 as described below.

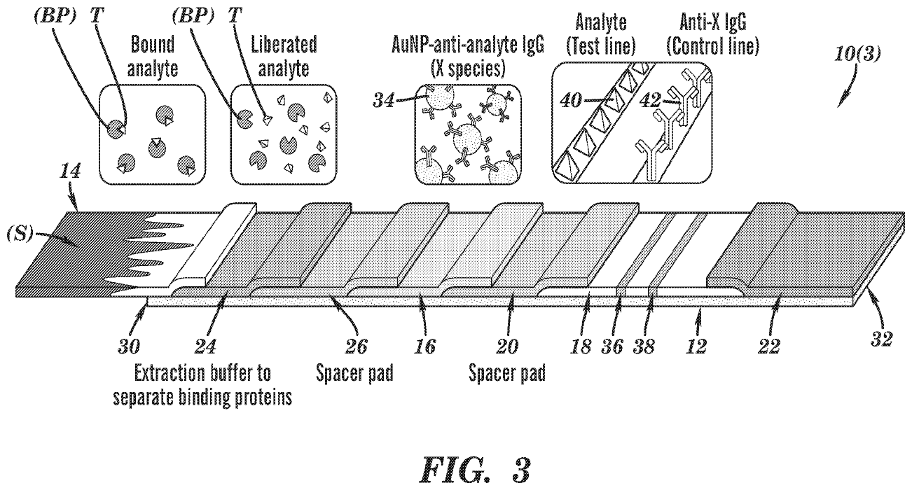

Referring now to FIG. 3, yet another embodiment of competitive diagnostic assay strip 10(3) is illustrated. Competitive diagnostic assay strip 10(3) is the same as competitive diagnostic assay strip 10(2) except as described below. Specifically, competitive diagnostic assay strip 10(3) includes an additional spacer layer after extraction layer 24 to allow for additional incubation time with between the target molecule (T) and the extraction buffer to allow for enhanced liberation of the target molecule (T) from the binding protein (BP).

Second spacer layer 26 is supported on substrate 12 downstream of extraction layer 24 and upstream of second layer 16. Second spacer layer 26 is made of glass fibers, although other suitable materials may be utilized. Second spacer layer 26 is designed to provide added mixing and incubation time for removal of the target molecule (T) with an extracting agent or buffer.

Second spacer layer 26 is designed to substantially stop flow of the material received from extraction layer 24 within second spacer layer 26 until a further fluid flow is received. By way of example, second spacer layer 26 may be configured in the same manner as first spacer layer 20. In order to restart the fluid flow between second spacer layer 26 and second layer 16, the user must introduce a volume of running buffer that fills the remaining volume of second spacer layer 26 to drive the sample (S) to second layer 18. The dimensions of second spacer layer 26, and in particular the volume capacity, determine the amount of additional fluid, such as a running buffer, that must be added to overflow second spacer layer 26 to deliver the sample (S) to second layer 16 for interaction with the conjugate stored therein. The ability to substantially stop the sample (S) in second spacer layer 26 allows for arbitrary incubation periods that may be determined by the user as described in further detail below. The delay provides added mixing and incubation time for the extracting agent 46 and the sample (S) and allows the user to actively control the incubation period.

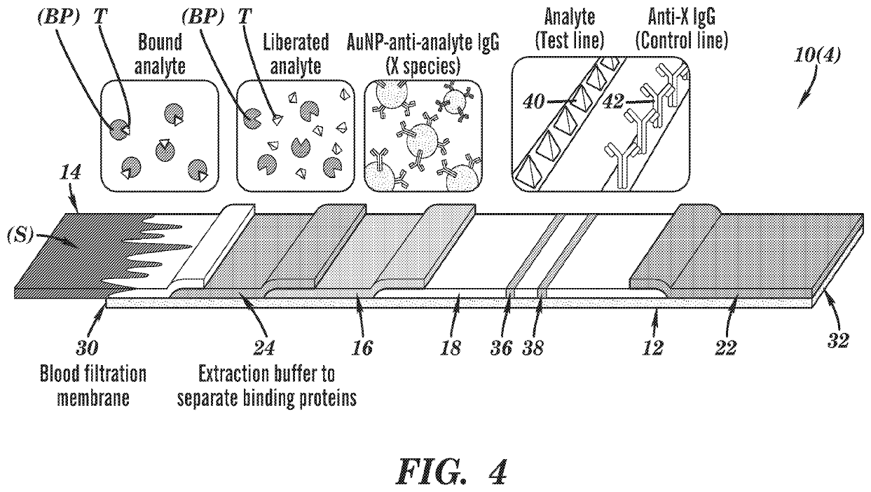

FIG. 4 is a perspective view of another embodiment of competitive diagnostic assay strip 10(4) of the present invention. Competitive diagnostic assay strip 10(4) is the same as competitive diagnostic assay strip 10(2) except as described below. Specifically, competitive diagnostic assay strip 10(4) includes extraction layer 24 but does not include any spacer layers.

Another aspect of the present invention relates to a diagnostic assay cartridge including the competitive diagnostic assay strip according to the present invention. The diagnostic assay cartridge includes an elongate housing having walls defining a chamber in which the competitive diagnostic assay strip is positioned. The cartridge extends between a first end proximate to the first end of the elongate substrate, where the sample is inserted through an inlet passage in a wall of the housing and into the chamber, and a second end proximate to the second end of the elongate substrate at which results of the assay can be assessed.

Referring again to FIG. 1A, competitive diagnostic assay strip 10(1) of the present invention may be housed in diagnostic assay cartridge 50, although other assay strips, such as competitive diagnostic assay strips 10(2)-10(4) could be utilized in diagnostic assay cartridge 50. Diagnostic assay cartridge 50 includes elongate housing 52 having walls 54 defining chamber 56 in which competitive diagnostic assay strip 10(1) is positioned. Chamber 56 is sized to receive competitive diagnostic assay strip 10(1) therein. Diagnostic assay cartridge 50 may be formed of any suitable materials for housing competitive diagnostic assay strip 10(1). Diagnostic assay cartridge 50 extends between first end 58 and second end 60. When completive diagnostic assay strip 10(1) is located in chamber 56, first end 58 is proximate to first end 30 of elongate substrate 12, while second end 60 is proximate to second end 32 of elongate substrate 12 at which results of the assay can be assessed. Diagnostic assay cartridge 50 includes inlet passage 62 that allows insertion of the sample (S) into chamber 56 and onto first layer 14. Diagnostic assay cartridge 50 also includes viewing portal 64 aligned with test region 36 and control region 38 to allow for analysis of the assay results, such as by imaging test region 36 and control region 38.

Yet another aspect of the present invention relates to a method of conducting a diagnostic assay. The method includes providing a competitive diagnostic assay strip in accordance with the present invention. A sample is applied to the first layer. A buffer is applied to the first layer after applying the sample to the first layer, whereby the buffer causes flow of material from the first end of the elongate substrate to the second end of the elongate substrate. The test and control regions in the third layer are analyzed to determine whether the target material is present in the sample and/or what quantity of the target material is present.

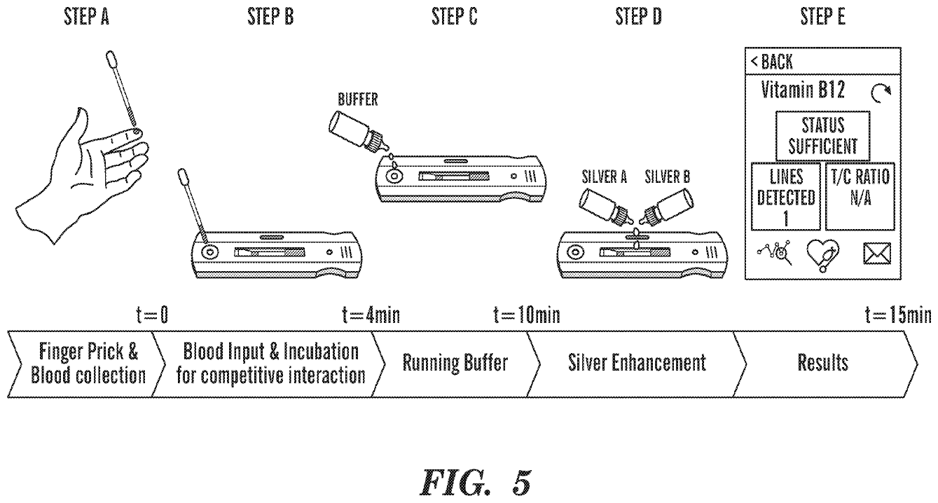

First, a competitive diagnostic assay strip according to the present invention is provided. In one example, competitive diagnostic assay strip 10(1) is provided, although the method may be utilized with other competitive diagnostic assay strips of the present invention, such as competitive diagnostic assay strip 10(2)-10(4). Referring now to FIGS. 1A-1C and 5, a method is described for a vitamin B₁₂ analysis of a blood sample using competitive diagnostic assay strip 10(1) located in diagnostic assay cartridge 50, although other target molecules may be analyzed for other fluid samples.

First, in step A, the user collects a raw blood sample (S) via a finger prick. In step B, the sample (S) is applied to inlet passage 62 in cartridge 50 which directs the sample (S) onto first layer 14 of competitive diagnostic assay strip 10(1) as shown in FIGS. 1A-1C. In one example, first layer 14 filters the blood sample (S) so that only the plasma migrates to second layer 16. In second layer 16, the vitamin B₁₂ in the sample (S) specifically binds to mobile labeled specific binding partner 34, which in this example is AuNP-anti-B₁₂ conjugate, to produce complex (C) of the vitamin B₁₂ molecules bound to mobile labeled specific binding partner 34. The complex (C) then flows to first spacer layer 20, which as described above has a volume capacity configured such that the input volume of the sample (S) will be insufficient to overflow first spacer layer 20. Thus, sample (S) does not reach third layer 18 without further user interaction. The ability to substantially stop the sample (S) in first spacer layer 20 allows for arbitrary incubation periods that may be determined by the user to allow sufficient binding interactions to occur.

In method step C, a running buffer is applied to first layer 14 through inlet passage 62 in cartridge 50 after applying the sample (S) to first layer 14. The running buffer may be applied after a sufficient incubation period to enhance the binding for optimal test results. In this example, the running buffer is applied by applying droplets from a dropper bottle after allowing approximately 4 minutes of incubation, although other incubation periods of any length may be utilized due to first spacer layer 20 stopping the flow of fluid. The incubation time may be selected to optimize the binding between the vitamin B₁₂ and mobile labeled specific binding partner 34. The running buffer is applied in a sufficient volume to cause first spacer layer 20 to overflow such that sample (S) including the formed complex (C) flows from first surface layer 20 to third layer 18. In test strips containing more than one spacer layer, such as competitive diagnostic assay strip 10(3), step C will be repeated as necessary to reinitiate the flow from the spacer layers.

Once in third layer, the sample (S) interacts with test region 36 and control region 38. As shown in FIG. 1B, for samples with high vitamin B₁₂ levels, most of the AuNP-anti-vitamin B₁₂ conjugates are occupied with vitamin B₁₂ molecules from the initial sample, and thus do not interact with immobilized target molecules 40, in this case vitamin B₁₂ molecules, located in test region 36, resulting in only a subtle colorimetric change in test region 36. The preoccupied AuNP-anti-vitamin B₁₂ that pass test region 36 without binding are captured by immobilized moiety 42 located in control region 38, resulting in a weak T/C signal intensity for high vitamin B₁₂ levels in sample (S). As shown in FIG. 1C, for samples with low vitamin B₁₂ levels, test region 36 develops an intense color reflecting the high number of AuNP-anti-vitamin B₁₂ that bind with immobilized target molecules 40 in test region. This leads to a weak signal in control region 38 due to the depleted number of AuNP-anti-vitamin B₁₂ reaching control region 38, and consequently strong T/C signal intensity for low vitamin B₁₂ levels in the sample (S).

Next, in step D, the complex (C) present in third layer 18 may optionally be amplified prior to analyzing test region 36 and control region 38. By way of example, a silver enhancement solution may be applied to enhance the colorimetric signals in test region 36 and control region 38. The silver enhancement may be applied after a delayed period of time, such as approximately 6 minutes, to allow for sufficient colorimetric development in test region 36 and control region 38 prior to amplifying the complex (C) present.

In step E, test region 36 and control region 38 are analyzed to determine whether the target material (T), such as vitamin B₁₂, is present in the sample (S). Test region 36 and control region 38 may be further analyzed to determine a quantity of the target material (T) present in the sample (S). Specifically, test region 36 and control 38 may be imaged and the obtained images processed based on the colorimetric signals to obtain a quantification of the target molecule (T) in the sample (S). In one example, test region 36 and control region 38 are analyzed using the methods described in U.S. Patent Application Publication No. 2016/0080548 and PCT Patent Application PCT/US14/12263, the disclosures of which are hereby incorporated by reference in their entirety herein, although other testing methods that employ image processing may be utilized such as commercial lateral assay flow readers, an example of which is the ESEQuant Lateral Flow Reader produced by Qiagen, Germany. Alternatively, the signal can be recorded over time optically with a camera, photomultiplier, or similar optical sensor. The images are then processed to provide a quantitative analysis of the amount of vitamin B₁₂ in the sample. The results of the method are then displayed, such as on the screen of a mobile computing device.

Referring now to FIG. 6, a method is described for a 25(OH) (vitamin D) analysis of a blood sample using competitive diagnostic assay strip 10(4) located in diagnostic assay cartridge 50, although other target molecules may be analyzed for other fluid samples.

First, in step A, the user collects a raw blood sample (S) via a finger prick. In step B, the sample (S) is applied to inlet passage 62 in cartridge 50 which directs the sample (S) onto first layer 14 of competitive diagnostic assay strip 10(3) as shown in FIG. 4. One of the main challenges in translating vitamin D diagnostics to a lateral flow format is the presence of the vitamin D binding protein. Vitamin D, being fat-soluble and hydrophobic, does not travel freely through the blood stream but is instead predominantly transported by the vitamin D binding protein (BP). In order to quantify vitamin D concentrations, therefore, it is necessary to first release the vitamin D from the binding protein (BP). In order to do this, an extraction buffer is also applied to extraction layer 24 through a second inlet in cartridge 50, although in other embodiments, the extraction buffer may be applied to an end of first layer 14. In this example, the extraction buffer comprises dimethyl sulfoxide and an acetate buffer. In one example, the dimethyl sulfoxide and a pH 4 acetate buffer are in an approximately 7:3 ratio. In one example the extraction buffer is applied in a 1:8 ratio with the volume of the sample (S) to be treated. First layer 14 filters the blood sample (S) so that only the plasma migrates to extraction layer 24. In extraction layer 24, the vitamin D interacts with the extracting agent which releases the vitamin D from the binding proteins (BP). The released vitamin D is then transmitted to second layer 16. In one example, second spacer layer 26 is located between extraction layer 24 and second layer 16, as shown in FIG. 3, to provide additional incubation time between the vitamin D and the extracting agent. Additional running buffer would then be required to pass the sample (S) through second spacer layer 26.

Next, in step C, a running buffer is applied to first layer 14 through inlet passage 62 in cartridge 50 after allowing sufficient time for the vitamin D to interact with the extracting agent. The running buffer is applied in a sufficient volume to cause the filtered plasma from sample (S) to flow to second layer 16.

In second layer 16, the vitamin D interacts with and specifically binds to mobile labeled specific binding partner 34, which in this example is AuNP-anti-D IgG conjugate, to produce complex (C) of the vitamin D molecules bound to labeled specific binding partner 34. In one embodiment, first spacer layer 20 is located after second layer 16 as shown in FIGS. 2 and 3 to provide additional incubation time as described above.

The sample (S) then flows to third layer 18, where the sample (S) interacts with test region 36 and control region 38. As shown in FIG. 1B, for samples with high vitamin D levels, most of the AuNP-anti-vitamin D conjugates are occupied with vitamin D molecules from the initial sample, and thus do not interact with immobilized target molecules 40, in this case vitamin D molecules, located in test region 36, resulting in only a subtle colorimetric change in test region 36. The preoccupied AuNP-anti-vitamin D that pass test region 36 without binding are captured by immobilized moiety 42 located in control region 38, resulting in a weak T/C signal intensity for high vitamin D levels in sample (S). For samples with low vitamin D levels, test region 36 develops an intense color reflecting the high number of AuNP-anti-D that bind with immobilized target molecules 40 in test region. This leads to a weak signal in control region 38 due to the depleted number of AuNP-anti-vitamin D reaching control region 38, and consequently strong T/C signal intensity for low vitamin D levels in the sample (S).

Next, in step D, test region 36 and control region 38 are analyzed to determine whether the target material (T), such as D, is present in the sample (S) as described above. Test region 36 and control region 38 may be further analyzed to determine a quantity of the target material (T) present in the sample (S). Specifically, test region 36 and control 38 may be imaged and the obtained images processed based on the colorimetric signals to obtain a quantification of the target molecule (T) in the sample (S). In one example, test region 36 and control region 38 are analyzed using the methods described in U.S. Patent Application Publication No. 2016/0080548 and PCT Patent Application PCT/US14/12263, the disclosures of which are hereby incorporated by reference herein, although other testing methods that employ image processing may be utilized such as commercial lateral assay flow readers, an example of which is the ESEQuant Lateral Flow Reader produced by Qiagen, Germany. The image processing provides for a quantitative analysis of the amount of vitamin D in the sample. The results of the method are then displayed, such as on the screen of a mobile computing device.

### EXAMPLES

### Example 1—Gold Nanoparticle (AuNP)-Anti-Vitamin B₁₂ Conjugate Pad Preparation

Monoclonal anti-vitamin B₁₂ immunoglobulin (IgG) produced in mouse (antibodies-online) in >95% purity was conjugated with 40 nm gold nanoparticles (AuNPs) using the InnovaCoat Gold Conjugation Kit (Innova Biosciences Ltd., United Kingdom). Briefly, 0.23 μg AuNP in freeze dried form was mixed with 1 pg anti-vitamin B₁₂ IgG in 0.01M amine-free phosphate buffer saline (PBS) buffer at pH 7.4. The anti-vitamin B₁₂ IgG attached stably to the surface of AuNP via lysine residues during the 15 min incubation and the reaction was terminated by adding 0.1M tris-buffered saline (TBS) with 0.1% Tween20.

To remove the excess antibody, 0.01M TBS with 0.1% Tween20 was added in 10 times the volume of the conjugate mixture and was centrifuged at 9000 g for 10 min. Upon removal of the supernatants, the final AuNP-anti-vitamin B₁₂ conjugates were reconstituted in 0.01M TBS containing 2% bovine serum albumin (BSA) and the final O.D. was checked using Spectramax 384 (Molecular Devices Corporation, Sunnyvale, Calif.) at 530 nm. The conjugates were stored at 4° C. until use. To obtain AuNP-anti-vitamin B₁₂ with varying conjugation ratios, the conjugation process was repeated using 0.75 and 0.50 μg anti-vitamin B₁₂ amounts. The different conjugation ratios lead to gold-antibody conjugates with different number of antibodies per gold particles, having an effect on the assay sensitivity by affecting the antibody-to-antigen binding events.

To prepare the conjugate pads for the vitamin B₁₂ assay, the AuNP-anti-vitamin B₁₂ conjugates were first diluted to 0.060 O.D. in the conjugate buffer (2 mM borate buffer with 5% sucrose). The Glass Fiber Conjugate Pads (EMD Millipore, Billerica, Mass.) with 30 cm×5 mm dimensions were soaked in the diluted conjugate solution for 1 min, followed by drying at 37° C. for 10 h.

### Example 2—Vitamin B₁₂ Lateral Flow Assay Preparation

Flow Plus 180 Membrane Cards (EMD Millipore, Billerica, Mass.) with a 2 mm clear polyester film backing were used as the assay platform, housing the nitrocellulose membrane and the adhesive parts where the conjugate, sample, and absorbent pads could be attached. Before the assembly, the test and control lines were prepared on the nitrocellulose membrane using the Lateral Flow Reagent Dispenser (Claremont Biosolutions, Claremont, Calif.) to dispense 0.325 mg/ml vitamin B₁₂-BSA conjugate (CalBioreagents Inc., San Mateo, Calif.) and 0.75 mg/ml anti-mouse IgG produced in goat (Sigma-Aldrich Co. LLC, St. Louis, Mo.), respectively. The two lines are separated by 3 mm and uniform line widths of 1 mm could be obtained by operating the Legato 200 Dual Syringe Pump (Claremont Biosolutions, Claremont, Calif.) at 6.4 μl/min.

The membrane cards were subsequently dried for 2 h at 37°, then at room temperature overnight. In order to investigate the effect of varying the vitamin B₁₂-BSA concentrations on the assay sensitivity, the coating process was repeated for 0.40, 0.35, 0.325, 0.30, and 0.25 mg/ml vitamin B₁₂-BSA. The vitamin B₁₂ lateral flow assay was assembled into its final form shown by first attaching the spacer (i.e., an untreated conjugate pad) to the adhesive region of the assay platform below the nitrocellulose membrane with an overlap of 0.5 mm. The AuNP-anti-vitamin B₁₂ treated conjugate pad was then attached below the spacer pad with 0.5 mm overlap.

The Fusion 5 Membrane (GE Whatman, St. Louis, Mo.) or FR-1 (MDI Membrane Technologies, India) was then attached below the AuNP-anti-vitamin B₁₂ conjugate pad with the 2 mm overlap to serve as the sample pad of the assay. While the Fusion 5 membrane works well with blood sample volumes below about 30 μl, the FR-1 (0.35) membrane has a larger capacity and is suitable for applications involving blood volumes above about 40 μl. The Cellulose Fiber Sample Pad (EMD Millipore, Billerica, Mass.) was attached above the nitrocellulose membrane with the 2 mm overlap to serve as the absorbent pad of the assay. The assembled assay was cut into individual strips of 4 mm width using a rotary paper trimmer (Dahle North America, Inc., Peterborough, N.H.).

### Example 3—Results for Vitamin B₁₂ Assay

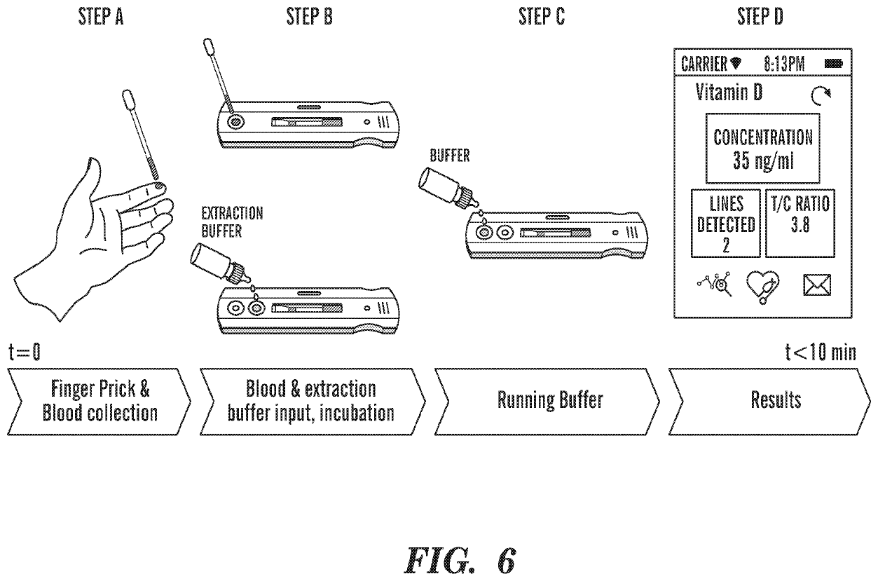

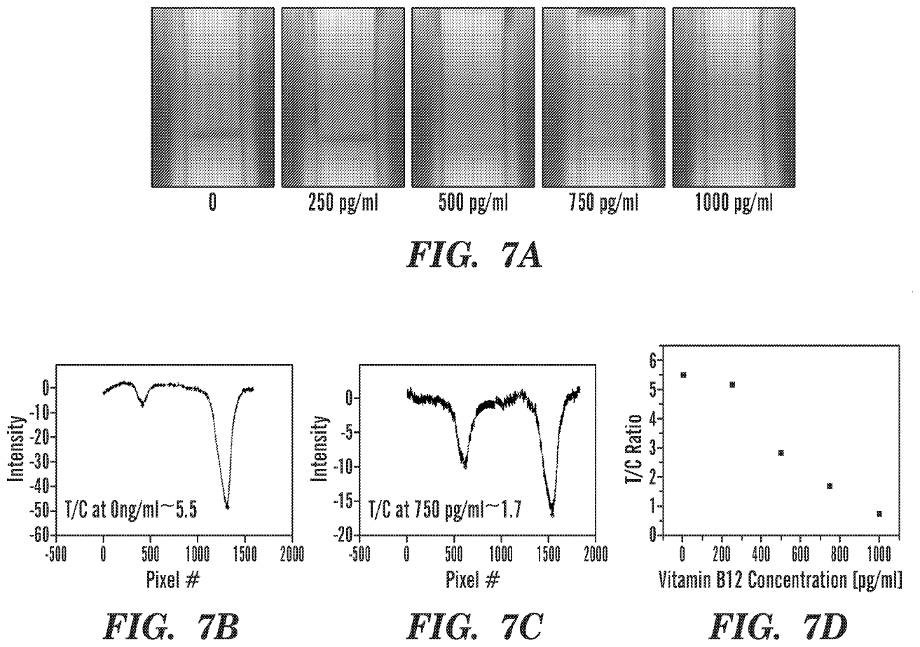

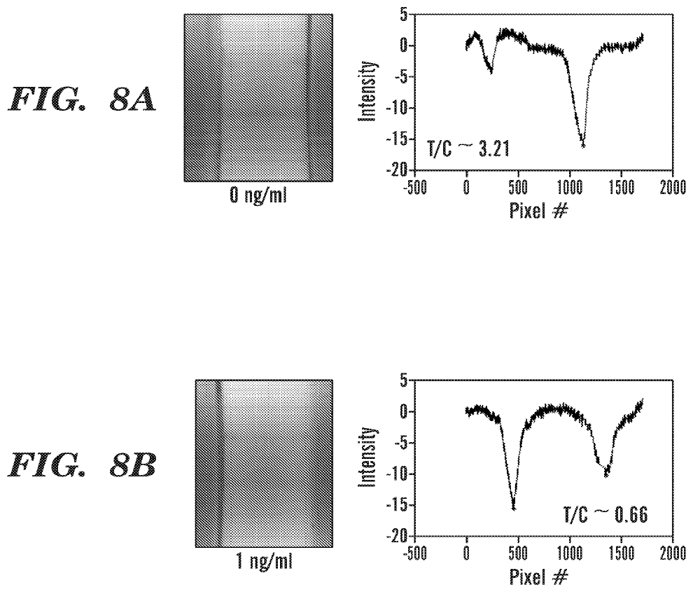

The vitamin B₁₂ assay, optimized initially with 0.35 mg/ml vitamin B₁₂-BSA to 0.060 OD gold anti-vitamin B₁₂, was first tested in standard vitamin B₁₂ calibrators in the 0-1000 pg/ml range. The colorimetric signals of the silver-enhanced vitamin B₁₂ assay were captured by an imaging system as shown in FIG. 7A, which demonstrates the expected trend of decreasing test-to-control line (T/C) ratio with increasing sample vitamin B₁₂. As shown in FIGS. 7B and 7C, the captured images could be analyzed for their T/C ratios using a computing platform. Furthermore, the vitamin B₁₂ strip images in the 0-1000 pg/ml could be analyzed to yield a calibration curve shown in FIG. 7D, which can be used for quantitative distinction in the sub ng/ml concentrations of sample vitamin B₁₂. In FIGS. 8A and 8B, the existence of multiple optimization points that can be used in the vitamin B₁₂ assay are demonstrated. Using different optimization conditions of 0.325 mg/ml vitamin B₁₂-BSA to 0.53 OD gold anti-vitamin B₁₂, the assay can still distinguish 0 from 1000 pg/ml as T/C quantification results indicated in FIGS. 8A and 8B.

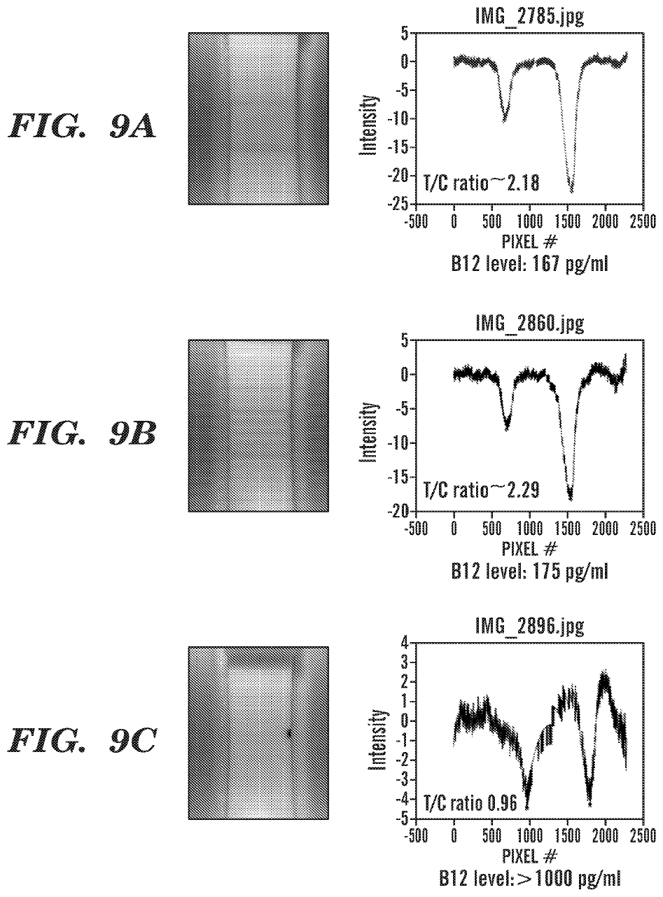

In initial human trials in which the vitamin B₁₂ assays were used to analyze finger prick blood samples, the “Nutriphone” system as described in detail in U.S. Patent Application Publication No. 2016/0080548 and PCT Patent Application PCT/US14/12263, the disclosures of which are hereby incorporated by reference in their entirety herein, successfully differentiated the vitamin B₁₂ deficient samples from the vitamin B₁₂ non-deficient sample as determined by the laboratory standard method (Immulite; Siemens Healthcare, Malvern, Pa.). As shown in FIGS. 9B and 9C, the whole blood samples from the participants with low vitamin B₁₂ was analyzed by the NutriPhone system to yield T/C ratios greater than 1, while the blood sample from the high vitamin B₁₂ participant yielded a T/C ratio less than 1.

### Example 4—Vitamin D Results

To demonstrate the efficacy of the lateral flow vitamin D assay, a series of human trials using human serum were conducted. In these tests, 40 μL of serum was added to the first sample inlet and the 5 μL of extraction buffer was added to the second inlet downstream. The extraction buffer comprises 3.5 μL of DMSO and 1.5 μL of pH 4.0 acetate buffer.

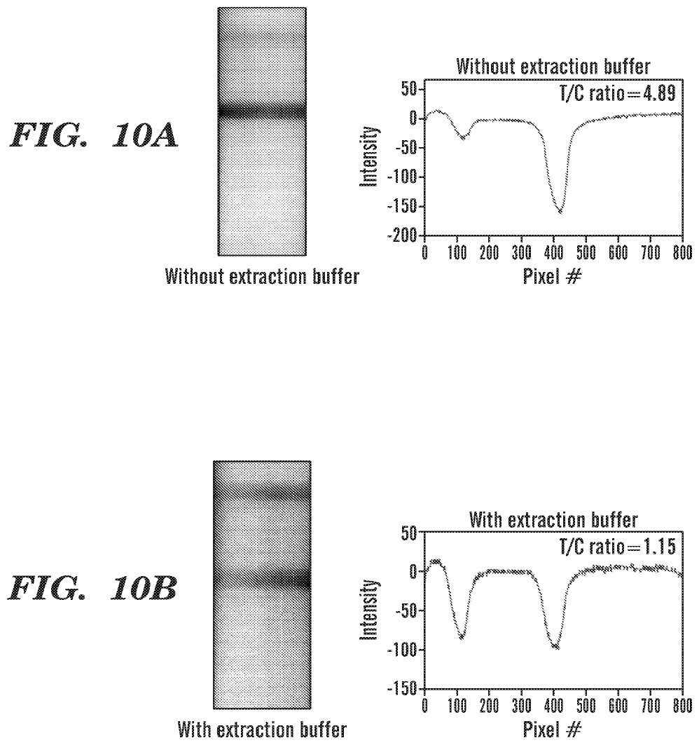

FIGS. 10A and 10B illustrate the effect of the extraction buffer for the vitamin D assay including the raw images and processed signals for a serum sample without extraction buffer (FIG. 10A) and with the extraction buffer (FIG. 10B). Without the extraction buffer present, the test line signal is very high relative to the control corresponding to very low vitamin D. This is due to the binding protein which prevents binding of the vitamin D to the gold nanoparticle conjugates. With the extraction buffer on the same sample, the test line signal decreases relative to the control due to the release of the vitamin D from the binding proteins which binds with the conjugates.

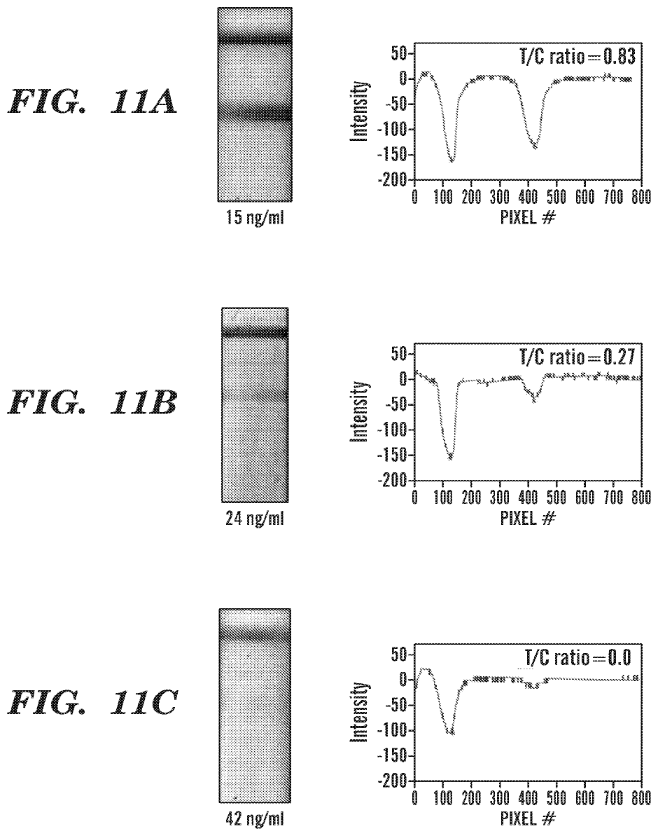

Three serum samples were collected with 25(OH)D (vitamin D) concentrations ranging from 15 ng/ml to 42 ng/ml, as verified by a commercial ELISA kit (Immunodiagnostic Systems, IDS, United Kingdom). As seen in FIG. 11A, there is a strong test line signal for the 15 ng/ml sample which decreases rapidly with concentration. The test line signal decreases with increasing concentration as illustrated in FIGS. 11B and 11C. The ratio of the test line signal to the control line signal (T/C ratio) is given for each of the three strips, which decreases from 0.83 at 15 ng/ml (FIG. 11A) down to 0 at 42 ng/ml (FIG. 11C).

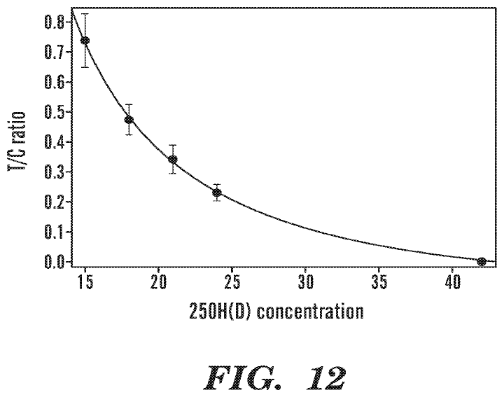

Imaging the device with an optical reader and applying an image processing algorithm, the T/C ratio for each sample is determined. From this, a full quantitative calibration curve is obtained, as shown in FIG. 12. This calibration is sensitive in the range of 15 ng/ml to 30 ng/ml which matches well with the standard cut-off of 20 ng/ml for 25(OH)D (vitamin D) insufficiency.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims that follow.

What is claimed:

1. A diagnostic system for detecting a target molecule in a liquid sample having a first volume, comprising:
an elongate substrate having a first end and a second end;
a first layer supported on the elongate substrate proximate to the first end for receiving, absorbing, and filtering the liquid sample;
a second layer comprising a conjugate pad supported on the elongate substrate downstream of the first layer for storing antibody conjugates;
a first spacer layer supported on the elongate substrate downstream of the second layer; and
a third layer supported on the elongate substrate downstream of the first spacer layer, the third layer comprising a test region and a control region, the first spacer layer defining a volume capacity such that: (i) the first volume of the liquid sample is insufficient to overflow the first spacer layer and (ii) a second volume of a buffer applied to the first layer, after applying the liquid sample to the first layer, is sufficient to cause the liquid sample to flow from the first spacer layer to the third layer.

2. The diagnostic system of claim 1 further comprising:
an extraction layer supported on the elongate substrate downstream of the first layer and upstream of the first spacer layer, wherein the extraction layer holds an extraction agent such that the liquid sample passing through the first layer interacts with the extracting agent, and wherein the extracting agent is configured to separate the target molecule in the liquid sample from a binding protein of the target molecule to permit recovery of the target molecule.

3. The diagnostic system of claim 2, wherein the extracting agent comprises dimethyl sulfoxide and an acetate buffer.

4. The diagnostic system of claim 1 further comprising:
an extraction buffer having a third volume to be applied to an extraction layer supported on the elongate substrate downstream of the first layer and upstream of the first spacer layer.

5. The diagnostic system of claim 4 further comprising:
a second spacer layer supported on the elongate substrate downstream of the extraction layer and upstream of the conjugate pad, the second spacer layer defining a second volume capacity such that: (i) the first volume of the liquid sample and the third volume of the extracting buffer are insufficient to overflow the second spacer layer and (ii) the second volume of the buffer applied to the first layer is sufficient to flow the liquid sample from the second spacer layer to the conjugate pad, the second volume of buffer is applied after applying the liquid sample to the first layer and after applying the extraction buffer to the extraction layer.

6. The diagnostic system of claim 1 further comprising:
an elongate housing having walls defining a chamber, wherein the housing comprises a first inlet configured to receive the liquid sample.

7. The diagnostic system of claim 6, wherein the elongate housing further comprises a second inlet positioned downstream of the first inlet.

8. The diagnostic system of claim 6, wherein the elongate housing further comprises a viewing portal aligned with the test region and the control region.

9. The diagnostic system of claim 1 further comprising:
a collection layer supported on the elongate substrate downstream of the third layer and proximate to the second end.

10. The diagnostic system of claim 1, wherein the second layer comprises a mobile labeled specific binding partner of the target molecule, wherein the mobile labeled specific binding partner is configured to bind specifically to the target molecule passing from the first layer through the second layer toward the second end.

11. The diagnostic system of claim 10, wherein the mobile labeled specific binding partner comprises a label selected from the group consisting of carbon nanoparticles, metallic nanoparticles, gold-nanoparticles, magnetic nanoparticles, fluorophores, quantum dots, and chemiluminescent particles.

12. The diagnostic system of claim 10, wherein the test region comprises an immobilized target molecule specifically binding to the complex of the target molecule bound to the mobile labeled specific binding partner, and immobilize the complex in the test region, and wherein the control region has an immobilized moiety which will non-specifically bind to the mobile labeled specific binding partner of the target molecule and immobilize it in the control region.

13. The diagnostic system of claim 1, wherein the third layer comprises two or more test regions and two or more control regions, the two or more test regions comprising different immobilized target molecules and the two or more control regions comprising different immobilized moieties which will non-specifically bind to different mobile labeled specific binding partners of the different target molecules to provide a multiplexed assay for different target molecules.

14. The diagnostic system of claim 1, wherein the target molecule is selected from the group consisting of vitamins, micronutrients, genetic biomarkers, carbohydrates, and proteins.

15. The diagnostic system of claim 1, wherein the first layer comprises a material selected from the group consisting of cellulose membranes, polyester matrix, glass fiber, and polysulfone membranes; wherein the second layer and the first spacer layer comprises glass fibers; and/or wherein the third layer comprises a material selected from the group consisting of cellulose and nitrocellulose.

16. The diagnostic system of claim 1 further comprising:
a signal enhancement solution configured to enhance colorimetric signals in the test region and the control region.

17. The diagnostic system of claim 1, wherein the diagnostic system is configured to measure a sub ng/ml concentration of a target molecule in the sample, and wherein the target molecule is vitamin B12.

18. The diagnostic system of claim 1 further comprising:
an optical sensor configured to record images of colorimetric signals in the test region and the control region;
one or more processors configured to provide a quantitative analysis of the target molecule in the liquid sample; and
a display interface configured to display the results of the quantitative analysis, wherein the results comprise a concentration of the target molecule.

19. A method of detecting a target molecule in a liquid sample having a first volume, comprising:
providing a diagnostic system of claim 1;
applying the liquid sample to said first layer, wherein the first volume of the sample is insufficient to overflow the first spacer layer;
applying the buffer to said first layer after said applying the liquid sample to said first layer, the buffer having a second volume sufficient to cause the liquid sample to flow from the first spacer layer to the third layer; and
analyzing the test and control regions in said third layer to determine whether the target material is present in the liquid sample and/or a quantity of the target material in the liquid sample.

20. A method of detecting a target molecule in a liquid sample having a first volume, comprising:
providing a diagnostic system comprising a diagnostic strip, the diagnostic strip comprising:
an elongate substrate having a first end and a second end;
a first layer supported on the elongate substrate proximate to the first end for receiving, absorbing, and filtering the liquid sample;
a second layer comprising a conjugate pad supported on the elongate substrate downstream of the first layer for storing antibody conjugates;
a first spacer layer supported on the elongate substrate downstream of the second layer; and
a third layer supported on the elongate substrate downstream of the first spacer layer, wherein the third layer comprises a test region and a control region;
applying the liquid sample to said first layer, wherein the volume of the liquid sample is insufficient to overflow the first spacer layer;
applying a buffer to said first layer after said applying the liquid sample to said first layer, the buffer having a second volume sufficient to cause the liquid sample to flow from the first spacer layer to the third layer; and
analyzing the test and control regions in said third layer to determine whether the target material is present in the liquid sample and/or a quantity of the target material in the liquid sample.
